(19) United States
(12) Patent Application Publication
(10) Pub. No.: US 2010/0312220 A1
(43) Pub. Date: Dec. 9, 2010

(54) SPIKE HAVING TWO PINS

(75) Inventor: Andreas Kalitzki, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: BAYER SCHERING PHARMA AKTIENGESELLSCHAFT, Berlin (DE)

(21) Appl. No.: 12/808,603

(22) PCT Filed: Dec. 4, 2008
(86) PCT No.: PCT/EP2008/010278

§ 371 Date: Jun. 16, 2010

(30) Foreign Application Priority Data
Dec. 17, 2007 (DE) 10 2007 061 346.8

(51) Int. Cl. A61M 5/162

(52) U.S. Cl. 604/411

(57) ABSTRACT

The invention relates to a removal device of an infusion system, with a holder part and with at least two penetrating parts protruding from the latter by different lengths, of which the shortest protruding penetrating part comprises a liquid channel, and of which the longest protruding penetrating part comprises at least an air channel. All the penetrating parts are arranged adjacent and parallel to one another. They have a shaft with a nominal cross-sectional area. Moreover, the inner cross-sectional area of the liquid channel is at least 60% of the greatest nominal cross-sectional area of the penetrating parts, at least in the region of the shaft.

With the present invention, a removal device is developed which permits safe piercing of the stopper and which ensures a substantial volumetric flow rate of the liquid, while maintaining the standard dimensions.

The invention relates to a removal device of an infusion system, with a holder part and with at least two penetrating parts protruding from the latter by different lengths, of which the shortest protruding penetrating part comprises a liquid channel, and of which the longest protruding penetrating part comprises at least an air channel.

The speed of infusions is limited by the volumetric flow rate from the infusion bottle, which is closed by means of a stopper, through the removal device into the drip chamber. The dimensions of the structural parts are subject to standards that ensure that the stopper is safely pierced through by the removal device.

A removal device is known from EP 1 652 544 A1. It comprises two penetrating parts which are arranged coaxially with respect to each other and of which the inner one comprises an air channel and the outer one comprises a liquid channel supplied from two containers. The volumetric flow rate of the liquid is limited by the cross section of the outer penetrating part, which is in turn defined by the geometry of the stopper. When the removal device is inserted, there is a danger of the stopper being broken out and of parts of the stopper falling into the infusion bottle.

The problem addressed by the present invention is that of developing a removal device which permits safe piercing of the stopper and which ensures a substantial volumetric flow rate of the liquid, while maintaining the standard dimensions.

This problem is solved by the features of the main claim. To this end, all the penetrating parts are arranged adjacent and parallel to one another. They have a shaft with a nominal cross-sectional area. Moreover, the inner cross-sectional area of the liquid channel is at least 60% of the greatest nominal cross-sectional area of the penetrating parts, at least in the region of the shaft.

Further details of the invention will become clear from the dependent claims and from the descriptions, given below, of the schematically illustrated embodiments.

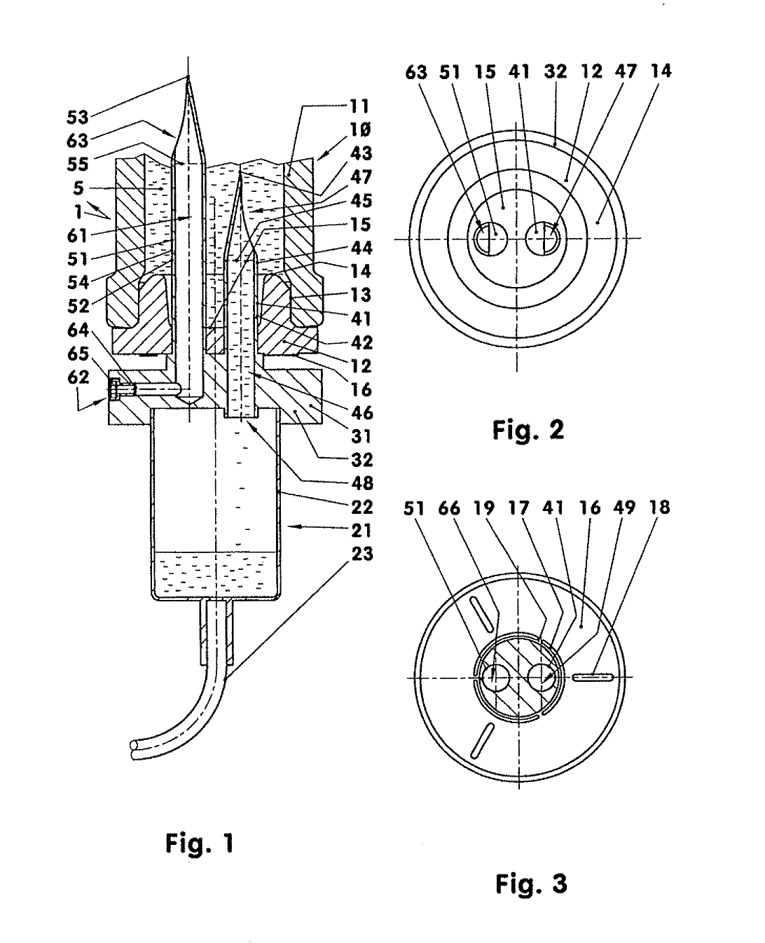

FIG. 1: section through an infusion bottle with stopper and removal device;

FIG. 2: plan view of the removal device from FIG. 1;

FIG. 3: section through the removal device from FIG. 1;

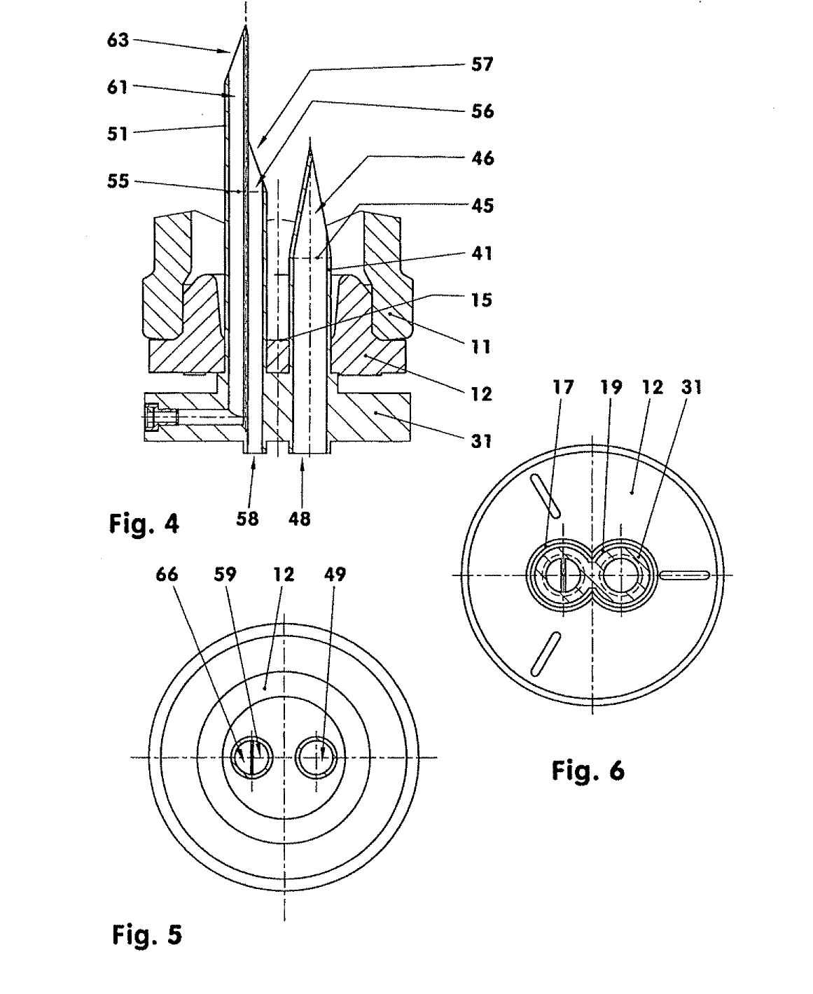

FIG. 4: assembled removal device with two liquid channels and one air channel;

FIG. 5: plan view of FIG. 4;

FIG. 6: removal device with modified stopper;

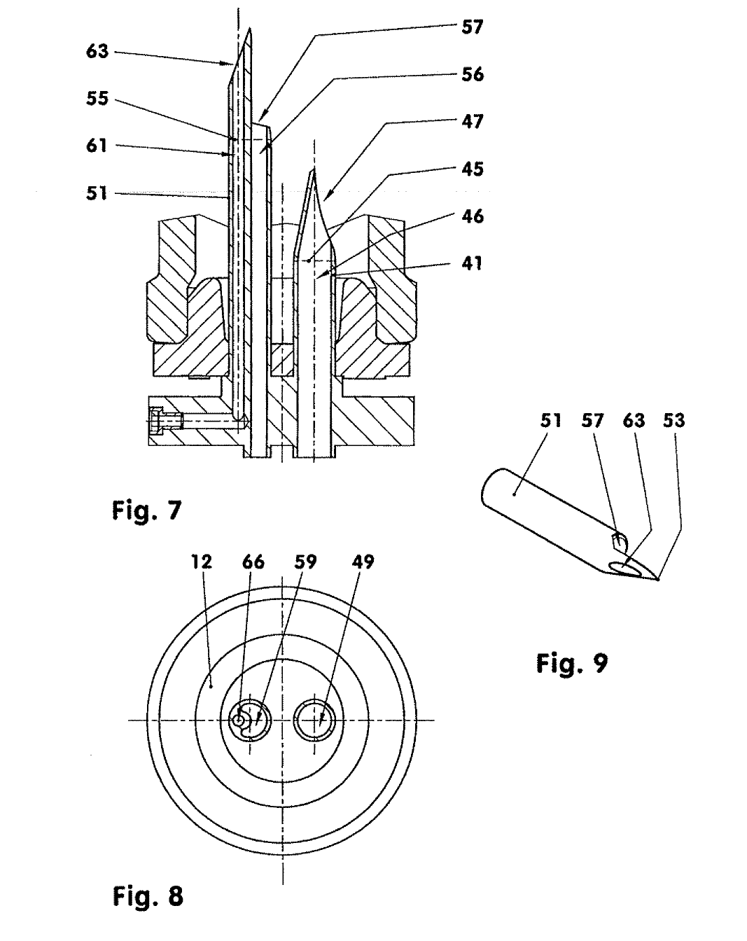

FIG. 7: removal device for high volumetric flow rate of liquid;

FIG. 8: plan view of FIG. 7;

FIG. 9: dimetric view of the longest protruding penetrating part in FIG. 7.

FIG. 1 shows in longitudinal section, as parts of an infusion system (10), an infusion bottle (11), with a stopper (12) inserted therein, and with a removal device (31) of an infusion set (21) engaged in the stopper (12).

To prepare for an infusion, the infusion bottle (11), which is filled with a liquid containing active substance, is first closed by means of the stopper (12). The stopper (12) is pierced by means of the removal device (31), and the infusion bottle (11) is then secured in a retainer, with the stopper (12) facing downward. The liquid (5) containing the active substance can now flow by gravity through the removal device (31) into the drip chamber (22), connected for example to the removal device (31), and into the infusion tube (23). A design without a drip chamber (22) is also conceivable, in which case the liquid (5) containing the active substance is sucked out of the infusion bottle (11) at a volumetric flow rate of, for example, up to 10 milliliters per second.

The infusion bottle (11) shown in FIG. 1 is, for example, a glass bottle and corresponds, for example, to the design described in EN ISO 8356-1, Form A. It here has a neck opening of 32 millimeters, for example. In this illustrative embodiment, the internal diameter at the upper edge is 22.5 millimeters.

The stopper (12) is, for example, a rubber stopper according to EN ISO 8536-2, Form A. In the unfitted state, it has an external diameter of 30.8 millimeters, for example, and a height of 12.2 millimeters. The diameter of the insert part (13), with which the stopper (12) is fitted into the infusion bottle (11), is 23.6 millimeters in the non-deformed state. On its top face (14) directed toward the infusion bottle (11), the stopper (12) has a recess (15) with a depth of, for example, eight millimeters, the bottom of said recess (15) having a diameter of 13 millimeters. The stopper (12), on its underside (16) (cf. FIG. 3), has a non-continuous reinforcement ring (17), which surrounds the surface of the recess (15) projected onto the underside (16). Additional reinforcement ribs (18) are arranged radially outside the reinforcement ring (17). They prevent the stoppers (12) from sticking during the transport and storage of the stoppers (12).

In the illustrative embodiment shown in FIG. 1 and in the plan view in FIG. 2, the removal device (31) comprises a holder part (32) and two penetrating parts (41, 51) which, for example, are formed integrally on the holder part (32). The penetrating parts (41, 51) can also be fitted into the holder part (32) or formed therein. The two penetrating parts (41, 51), for example penetrating pins (41, 51), are arranged adjacent and parallel to each other. The penetrating pin (41) shown here on the right, and referred to hereinafter as the short penetrating pin (41), has a free length of, for example, 28 millimeters protruding upward from the holder part (32). The long penetrating pin (51), shown on the left, protrudes from the holder part (32) by, for example, 43 millimeters.

In the illustrative embodiment, both penetrating pins (41, 51) have a maximum external diameter of 5.6 millimeters. They each protrude from the holder part (32) with a for example cylindrical or conical shaft (42, 52) and with a tip (43, 53) directed away from the holder part (32). At the transition to the tip (43, 53), the respective shaft (42, 52) has a circular cross section with a diameter of 5.2 millimeters, for example. These cross-sectional areas (45, 55) are referred to hereinbelow as nominal cross-sectional areas (45, 55). They are indicated in FIG. 1, for example, by a broken line. In FIG. 2, they are defined, for example, by the circumferential lines of the penetrating pins (41, 51). The distance of the nominal cross-sectional areas (45, 55) from the upper end of the tips (43, 53) is in each case, for example, 13 millimeters.

The short penetrating pin (41) extends through the holder part (32). It has a longitudinal channel (46) with a constant cross section or with a cross section that widens from the top downward. With a wall thickness of 0.5 millimeter, for example, the maximum inner cross section of this liquid channel (46) is 65% of the nominal cross-sectional area (45) of the penetrating pin (41). The inlet opening (47) of the liquid channel (46), located at the top in FIG. 1, is part of the circumferential surface (44) and adjoins, for example, the tip (43).

The long penetrating pin (51) has a longitudinal channel (61) which, within the holder part (32), is diverted outward in a radial direction. In the region of the nominal cross-sectional area (55), for example, this air channel (61) has the same inner cross-sectional area (66) as the liquid channel (46). At its inlet opening (62) on the holder part, it has, for example, a semipermeable membrane (64) and a bacteria-proof air filter (65). The outlet opening (63) of the air channel (61), located at the top in FIG. 1, is part of the circumferential surface (54) and adjoins, for example, the tip (53) of the long penetrating pin (51).

In order to connect the infusion set (21) to the infusion bottle (11), the removal device (31) is first applied to the stopper (12). In doing this, the long penetrating pin (51) first makes contact with the pierceable region (19) of the stopper (12) delimited by the reinforcement ring (17). As it is pressed into the stopper (12), the tip (53) of the long penetrating pin (51) cuts and pushes aside the material of the stopper (12). As soon as the long penetrating pin (51) in this illustrative embodiment has pierced through the stopper (12), the short penetrating pin (41) makes contact with the pierceable region (19) of the stopper (12). As the removal device (31) is pressed in farther, the short penetrating pin (41) also pierces through the stopper (12), cf. FIG. 3. Here, the surface of the stopper (12) pierced through by the nominal cross-sectional areas (45, 55) of the penetrating pins (41, 51) is 5.6% of the projected surface of the underside (16) of the stopper (12) or 32% of the surface of the pierceable region (19). In this illustrative embodiment, the inner cross-sectional areas (49, 66) of the liquid channel (46) and air channel (61), respectively, are therefore each 10.5% of the projected surface of the pierceable region (19).

After the infusion bottle (11) has been hung up, the tip (43) of the short penetrating pin (41) protrudes into the liquid (5) by 24 millimeters for example, whereas the long penetrating pin (51) protrudes into the infusion bottle (11) by 39 millimeters for example.

At the start of the infusion, the liquid (5) present in the infusion bottle (11) flows by gravity through the liquid channel (46) into the drip chamber (22). At the same time, air from the environment (1) flows through the air filter (65), the membrane, (64) and the air channel (61) into the infusion bottle (11). A high volumetric flow rate of the liquid (5) is achieved by virtue of the large cross section of the liquid channel (46) and by virtue of a sufficient supply of air through the air channel (61).

The penetrating parts (41, 51) can also have different nominal cross-sectional areas (45, 55). For example, the penetrating part (41) with the liquid channel (46) has a greater nominal diameter than the penetrating part (51) with the air channel (61). At least in the area of the shaft (42), the inner cross-sectional area (49) of the liquid channel (46) is then at least 60% of the greater nominal cross-sectional area (45, 55) of the for example two penetrating parts (41, 51).

FIGS. 4 and 5 show another illustrative embodiment of a removal device (31), respectively in section and in a plan view. The structure of the short penetrating pin (41) is as described in connection with FIGS. 1 to 3. The long penetrating pin (51) has an air channel (61) and also a liquid channel (56), which are both arranged parallel to each other. The inlet opening (57) of the liquid channel (56) lies 14 millimeters, for example, below the outlet opening (63) of the air channel (61). This liquid channel (56) extends through the holder part (32), its lower end lying, for example, at the same height as the lower end of the liquid channel (46) of the short penetrating pin (41). In this illustrative embodiment, the air channel (61) has the same length as the air channel (61) shown in FIG. 1.

In the illustrative embodiment shown in FIGS. 4 and 5, the liquid channel (56) and air channel (61) of the long penetrating pin (51) have an identical inner cross-sectional area (59, 66). This is in each case 35%, for example, of the nominal cross-sectional area (45, 55) of a penetrating part (41, 51). Thus, in this illustrative embodiment, the total cross-sectional area of the liquid channels (46, 56) is 100% of the nominal cross-sectional area (45, 55) of a penetrating pin (41, 51).

Since the cross section of the air channel (61) is not particularly critical during an infusion, the removal device (31) shown here permits a still greater volumetric flow rate of liquid compared to the variant shown in FIGS. 1 to 3.

FIG. 6 shows a cross section through a removal device (31) from FIGS. 4 and 5 and through a modified stopper (12). The reinforcement ring (17) and therefore the pierceable region (19) surrounded by it are shaped like spectacles. If appropriate, the stopper recess (15) can also have this shape. When this stopper (12) is pierced through, there is less danger of the pierceable region (19) tearing. With the dimensions of the penetrating pins (41, 51) shown in FIGS. 4 and 5, the sum of the inner cross-sectional areas (49, 59) of the liquid channels (46, 56) is 22% of the surface of the pierceable region (19). Relative to the projected surface of the underside (16) of the stopper (12), the sum of the inner cross-sectional areas (49, 59) of the liquid channels (46, 53) is 3% of the projected stopper surface.

FIGS. 7 and 8 show a further example of a removal device (31). The short penetrating pin (41) corresponds to the penetrating pins (41) shown in FIGS. 1 and 4.

The length dimensions of the long penetrating pin (51) correspond to the dimensions of the long penetrating pin (51) shown in FIGS. 4 and 5. In this illustrative embodiment too, the long penetrating pin (51) has an air channel (61) and a liquid channel (56).

The cross-sectional area (59) of the liquid channel (56) is kidney-shaped in this illustrative embodiment. It makes up 42% of the nominal cross-sectional area (45, 55) of a penetrating pin (41, 51). The sum of the inner cross-sectional areas (49, 59) of the liquid channels (41, 51) is therefore 107%, for example, of the nominal cross-sectional area (45, 55) of a penetrating pin (41, 51).

The air channel (61) has, for example, a round cross section. The cross-sectional area (66) of the air channel (61) in this removal device (31) is in this case 7% of the nominal cross-sectional area (45, 55).

FIG. 9 shows a dimetric view of the long penetrating pin (51) from FIGS. 7 and 8, without the holder part (32). The air outlet (63) and the tip (53) conceal part of the liquid inlet (57) in this view.

In order to achieve a still greater flow of liquid, the use of three or more penetrating parts (41, 51) is also conceivable. In this case, for example, the longest penetrating part (51) has an air channel (61), and all the other penetrating parts (41) each have a liquid channel (46). However, the penetrating parts (41, 51) can also be configured such that all of them have a liquid channel (46, 56). The longest penetrating part (51) then additionally comprises an air channel (61).

### LIST OF REFERENCE SIGNS

1 environment
5 liquid
10 infusion system
11 infusion bottle
12 stopper
13 insert part
14 top face
15 recess
16 underside
17 reinforcement ring
18 reinforcement ribs
19 pierceable region
21 infusion set
22 drip chamber
23 infusion tube
31 removal device, piercing device
32 holder part
41 shortest protruding penetrating part, short penetrating part, penetrating pin
42 shaft
43 tip
44 circumferential surface
45 nominal cross-sectional area
46 longitudinal channel, liquid channel
47 inlet opening
48 outlet opening
49 inner cross-sectional area
51 longest protruding penetrating part, long penetrating part, penetrating pin
52 shaft
53 tip
54 circumferential surface
55 nominal cross-sectional area
56 longitudinal channel, liquid channel
57 inlet opening
58 outlet opening
59 inner cross-sectional area
61 longitudinal channel, air channel
62 inlet opening
63 outlet opening
64 membrane
65 filter, air filter
66 inner cross-sectional area

1. A removal device (31) of an infusion system (10), with a holder part (32) and with at least two penetrating parts (41, 51) protruding from the latter by different lengths, of which the shortest protruding penetrating part (41) comprises a liquid channel (46), and of which the longest protruding penetrating part (51) comprises at least an air channel (61), characterized in that
all the penetrating parts (41, 51) are arranged adjacent and parallel to one another,
all the penetrating parts (41, 51) have a shaft (42, 52) with a nominal cross-sectional area (45, 55), and
the inner cross-sectional area (49) of the liquid channel (46) is at least 60% of the greatest nominal cross-sectional area (45, 55) of the penetrating parts (41, 51), at least in the region of the shaft (42).

2. The removal device (31) as claimed in claim 1, characterized in that the nominal cross-sectional area (45, 55) of the penetrating parts (41, 51) is at least almost identical.

3. The removal device (31) as claimed in claim 1, characterized in that the inner cross-sectional area (49) of the liquid channel (46) corresponds to the inner cross-sectional area (66) of the air channel (61).

4. The removal device (31) as claimed in claim 1, characterized in that it comprises at least two liquid channels (46, 56), and the sum of the inner cross-sectional areas (49, 59) is greater than the nominal cross-sectional area (45, 55) of one penetrating part (41, 51).

5. The removal device (31) as claimed in claim 1, characterized in that the longest protruding penetrating part (51) additionally comprises a liquid channel (56).

6. The removal device (31) as claimed in claim 3, characterized in that the two liquid channels (46, 56) have different lengths.

7. The removal device (31) as claimed in claim 1, characterized in that the air channel (61) has a filter (65).
